(19) United States
(12) Patent Application Publication
(10) Pub. No.: US 2020/0038119 A1
(43) Pub. Date: Feb. 6, 2020

(54) SYSTEM AND METHOD FOR TRAINING AND COLLABORATING IN A VIRTUAL ENVIRONMENT

(71) Applicant: Surgical Theater LLC, Mayfield Village, OH (US)

(72) Inventors: Alon Yakob Geri, Beachwood, OH (US); Mordechai Avisar, Highland Heights, OH (US)

(21) Appl. No.: 16/340,324

(22) PCT Filed: Mar. 23, 2018
(86) PCT No.: PCT/US2018/024154

§ 371 Date: Apr. 8, 2019

Related U.S. Application Data
(60) Provisional application No. 62/476,259, filed on Mar. 24, 2017.

(51) Int. Cl. A61B 34/00; G06F 3/14; G06F 3/0346; G06T 19/00; H04L 29/06; H04L 12/24; G16H 70/00; G16H 30/40; G16H 50/50; G16H 80/00; G16H 40/67; G06F 16/53; A61B 34/10; A61B 90/50; G06F 3/01

(52) U.S. Cl. CPC A61B 34/25; G09B 23/28; G06F 3/0346; G06T 19/003; H04L 65/403; H04L 41/0806; G16H 70/00; G16H 30/40; G16H 50/50; G16H 80/00; G16H 40/67; G06F 16/53; A61B 34/10; A61B 90/50; G06F 3/011; G06T 2210/41; G06T 2219/024; A61B 2034/105; A61B 2034/104; A61B 2090/502; A61B 2034/254; A61B 2034/107; A61B 2034/252; A61B 2034/256; G06F 3/1454

(57) ABSTRACT

A system for facilitating a collaboration includes a database for storing content. The system further includes a plurality of head mounted displays. The system further includes a computer server comprising one or more processors, one or more computer-readable tangible storage devices, and program modules stored on at least one of the one or more storage devices for execution by at least one of the one or more processors. The program modules include a first program module for retrieving the content from the database. The program modules further include a second program module for synchronously delivering the content to the plurality of head mounted displays. The program modules further include a third program module for receiving data representative of an interaction with the content. The program modules further include a fourth program module for synchronously delivering updated content to the plurality of the head mounted displays based on the received interaction.

### CROSS-REFERENCE TO RELATED APPLICATIONS

This application is a national stage application of PCT application serial number PCT/US2018/024154 filed on Mar. 23, 2018, which claims priority from U.S. provisional patent application Ser. No. 62/476,259 filed on Mar. 24, 2017, both of which are incorporated by reference herein in its entirety.

### FIELD OF DISCLOSURE

The present disclosure relates to field of training and collaboration and more particular to a system and method for training and collaborating in a virtual environment.

### BACKGROUND

Certain surgical procedures may be complex and therefore may require specific training and extensive planning and preparation. For example, during the course of high-risk surgeries such as cerebral aneurysm repair surgeries, the absolute orientation of the brain tissue is significantly altered as a surgeon pushes and cuts tissues to approach the aneurysm area. Further, surgeries, such as aneurysm repair, are extremely time-sensitive, due to various procedures including temporary vessel clamping to the aneurysm area. Therefore, the accuracy and efficiency of the procedure is highly critical and detailed planning based on the patient specific local geometry and physical properties of the aneurysm are fundamental.

A surgery rehearsal and preparation tool previously described in U.S. Pat. No. 8,311,791, incorporated in this application by reference, has been developed to convert static CT and Mill medical images into dynamic and interactive multi-dimensional full spherical virtual reality, six (6) degrees of freedom models (“MD6DM”) that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional 2 dimensional patient medical scans, that gives spherical virtual reality 6 degrees of freedom (i.e. linear; x, y, z, and angular, yaw, pitch, roll) in the entire volumetric spherical virtual reality model.

The MD6DM is built from the patient's own data set of medical images including CT, MRI, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved, and can be appreciated using the MD6DM.

The algorithm of the MD6DM takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD6DM reverts it to a 3D model by representing a 360° view of each of those points from both the inside and outside.

It may be desirable for multiple medical professionals, students, and other actors to participate in such surgical training and perpetration in a collaborative manner. Tools such as the surgery rehearsal and preparation tool described may not be capable of efficiently and effectively fostering such collaboration of multiple actors.

### SUMMARY

A system for facilitating a collaboration includes a database for storing content. The system further includes a plurality of head mounted displays. The system further includes a computer server comprising one or more processors, one or more computer-readable tangible storage devices, and program modules stored on at least one of the one or more storage devices for execution by at least one of the one or more processors. The program modules include a first program module for retrieving the content from the database. The program modules further include a second program module for synchronously delivering the content to the plurality of head mounted displays. The program modules further include a third program module for receiving data representative of an interaction with the content. The program modules further include a fourth program module for synchronously delivering updated content to the plurality of the head mounted displays based on the received interaction.

A method for facilitating a collaboration includes a computer retrieving content from a database. The method further includes the computer synchronously delivering the content to a plurality of head mounted displays. The method further includes the computer receiving data representative of an interaction with the content. The method further includes the computer synchronously delivering updated content to the plurality of the head mounted displays based on the received interaction.

A system for facilitating a collaboration includes a plurality of head mounted displays. The system further includes a computer server comprising one or more processors, one or more computer-readable tangible storage devices, and program modules stored on at least one of the one or more storage devices for execution by at least one of the one or more processors, the program modules. The program modules include a first program module for receiving data content representative of a virtual environment. The program modules further include a second program module for synchronously delivering the content to the plurality of head mounted displays. The program modules include a third program module for receiving data representative of a movement in the virtual environment. The program modules include a fourth program module for synchronously delivering updated content to the plurality of the head mounted displays based on an updated perspective of view of the virtual environment associated with the movement.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example virtual stadium system.

FIG. 2 illustrates an example virtual SNAP computer.

FIG. 3 illustrates an example virtual stadium system.

FIG. 4 illustrates an example virtual operating room in an example virtual stadium.

FIG. 5 illustrates an example virtual stadium system.

FIG. 6 illustrates an example virtual stadium system.

FIG. 7 illustrates an example virtual 3D model display.

### DETAILED DESCRIPTION

The following acronyms and definitions will aid in understanding the detailed description:

AR—Augmented Reality—A live view of a physical, real-world environment whose elements have been enhanced by computer generated sensory elements such as sound, video, or graphics.

VR—Virtual Reality—A 3Dimensional computer generated environment which can be explored and interacted with by a person in varying degrees.

HMD—Head Mounted Display refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Case—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Avatar—An avatar represents a user inside the virtual environment.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

Described herein is a system and method for facilitating training and collaboration in a virtual environment. The system enables multiple users, including an instructor and participants, to interact with various types of content in a virtual environment in real time. Content may include, for example, a 3D model of an entire patient, a 3D model of an organ, a virtual operating room, and a virtual library. The instructor may move around the 3D patient model, go inside the patient's 3D body, pick up 3D model organs for closer inspection, move around a virtual operating room, perform a virtual surgical procedure inside the virtual operating room, or engage with content in the virtual library, for example. As the instructor navigates the content, the participants are shown the same content in sync with the instructor so that the participants can follow along to learn and collaborate. The participants may be given some autonomy with respect to movement around and within the content, as represented by individual avatars, such that each participant may be able to have a unique and personal perspective and experience while still following along with the instructor and the other participants. The instructor may make notes, add drawings, provide audio commentary, etc. during a training and collaboration session, which the participants can see in real time.

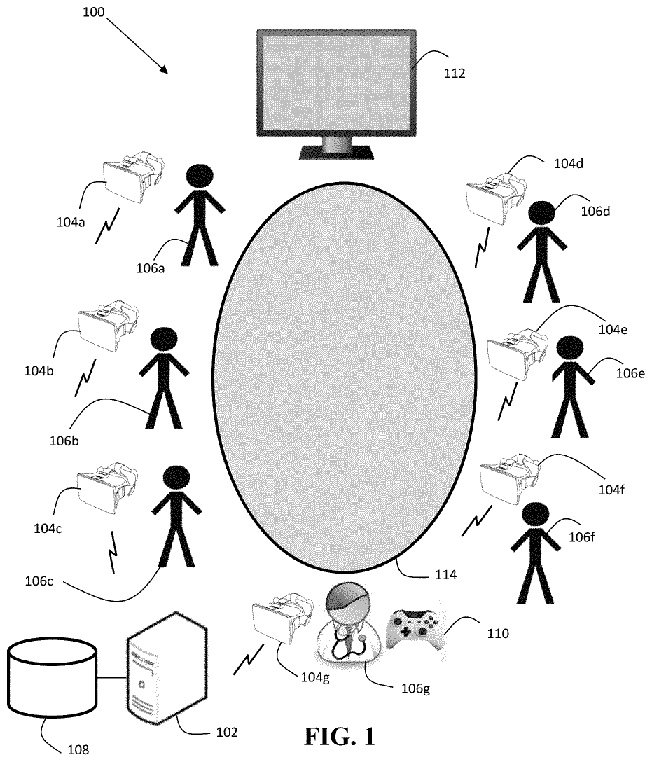

A virtual stadium system 100 for enabling a virtual environment for training and collaborating (herein after referred to as a “virtual stadium” or “VR stadium”) 114 is illustrated in FIG. 1. The virtual stadium 114 enables multiple people to interact in the virtual stadium 114 in order to learn from an instructor and from each other, and also to collaborate together on solving specific problems. For example, physicians or other instructors may interact with students in the virtual stadium 100 in order to train users on specific medical procedures. Physicians may also interact with other physicians or other specialists in a virtual stadium 100 in order to collaborate on treating a patient.

The VR stadium system 100 includes a VR stadium server 102 comprising hardware and specialized software that can be executed on hardware that generates and facilitates the VR stadium 114. In particular, the VR stadium server 102 communicates with one or more head mounted displays 104a-104g (hereinafter referred to as “HMD” 104) in order to deliver content to, as well as receive data from, one or more users 106a-106g (hereinafter referred to as user 106) via the HMD 104. The VR stadium server 102 retrieves content from a VR stadium database 108 in order to deliver to the HMD 104.

It should be appreciated that content retrieved from the VR stadium database 108 may include any suitable type of content for training and collaborating on various types of medical conditions and procedures. This content may include images and medical parameters of organs or other tissues that are obtained from one or more particular patients via medical imaging procedures, such as discussed in U.S. Pat. No. 8,311,791 filed on Oct. 19, 2010, and incorporated herein by reference, where it is discussed that medical images of a particular patient (e.g., CT scans, MM, x-rays, etc.) are converted into realistic images of that particular patient's real organs with surrounding tissues and any defects. This content may also include images and parameter related to real surgical or other medical tools used by physicians for performing actual medical procedures in patients. In particular, once content is delivered to the HDMs 104, the group of users 106 may visualize, discuss, provide input, receive feedback, and learn from one another while all being immersed in the same virtual stadium 114.

In one example, a head or lead user such an instructor 106g may be given control of interaction with and navigating through the delivered content in order to lead a discussion or training session. In such an example, the other users 106 all see the same content from the same perspective as the instructor 106g via their respective HDMs 104. The instructor 106g has a handheld controller 110 that the instructor 106g uses to navigate through the virtual content. It should be appreciated that the instructor 106g may also navigate through the virtual content using gestures or using any other suitable means for navigating and manipulating virtual content and objects. The rest of the users 106, some or all of whom may be located remotely from the instructor 106g as in another room, or even another geographical location, or in diverse locations, follow along and see the content through which the instructor 106g is navigating. The instructor may also use the handheld controller 110 to make notes, marks, drawings, and so on, which the other users 106 will also see via their respective HDMs 104. The VR stadium server 102 synchronizes the content delivered to each HDM 104 to ensure that each user 106 is seeing the same content at the same time that the instructor 106g is seeing, including any notes, marks, etc.

In one example, each user 106 may have his or her own controller (not shown). In such an example, a user 106 may have autonomy to move around the virtual stadium 114 freely. In one example, a user 106 may move around a virtual stadium 114 but may be restricted to certain functions or content based on restrictions imposed by the instructor 106g. For example, an instructor 106g may give users permission to navigate to certain virtual content in a virtual stadium 114 only after the instructor 106g has first navigated to the same virtual content.

In another example, a user 106 may create notes which might include text and/or drawings and/or graphical images to share with the other users 106, which may further encourage collaboration and learning. In one example, the instructor 106g may limit the types of notes and input that a user 106 may share and may also limit the timing of when such notes and input may be shared. For example, an instructor may limit the users 106 to creating input such as notes, via their own controllers (not shown) to only when the instructor 106g stops talking and asks for input or questions. The instructor 106g may also chose to either allow the input from a specific user 106 to immediately be synchronized with all of the other users' 106 content and delivered to all HDMs 104 or the instructor 106g may choose to have such input delivered to only his own HDM 104g. The VR stadium server 102 is responsible for implementing any appropriate rules and restrictions and synchronizing content delivered to each HDM 104 accordingly.

It should be appreciated that the virtual stadium system 100 may include other features for enabling navigation in the virtual stadium 114 and for providing input and feedback. For example, even though a controller 110 has been described for navigating through the virtual stadium 114, one example virtual stadium system 100 may include sensors (not shown) for tracking a user's 106 movement. For example, one or more sensors positioned on the HDM 104 may track a user's 106 head movement and communicate such movement to the VR stadium server 102. The VR stadium server 102 may then use such sensor information to determine the virtual content to be delivered to the respective HDM 104. In another example, sensors placed inside a physical room may track a user's 106 physical movement and communicate such information to the VR stadium server 102, which may then deliver virtual content to the user's 106 HDM 104 accordingly.

In one example, the VR stadium system 100 may further include microphones (not shown) to enable users 106 to provide audible feedback to the stadium server 102 which may then be shared with the other users 106 and synchronized with distributed virtual content. These audio recordings may be electronically recorded for future playback.

The VR stadium 100 further includes a display 112b for displaying content as experienced by the instructor 106g via the HDM 104g. Thus, additional users who may not have access to an HDM 104 may still see the content and follow along and participate via one or more displays 112b. It should be appreciated the display 112 may be located either within physical proximity of the instructor 106g or in a remote location.

It should be appreciated that VR stadium server 102 may communicate with the HDMs 104, the controller 110, the display 112, and with other suitable components either wirelessly, such as by WiFi or Bluetooth, for example, or via wired connection such as Ethernet, for example.

It should be appreciated that, although the example VR stadium system 100 may described with specific references to training and collaborating in the medical field, the VR stadium system 100 may similarly be used in other fields in order to enable a variety of types of professionals to train and collaborate.

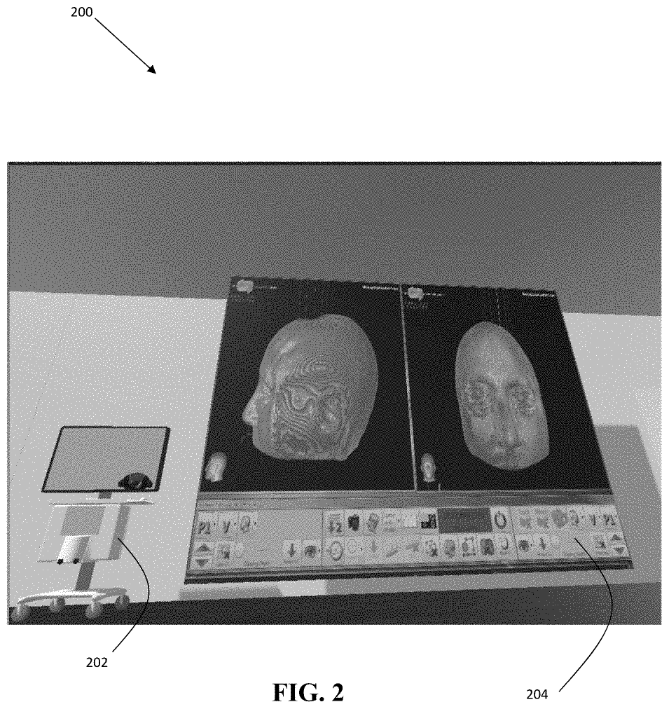

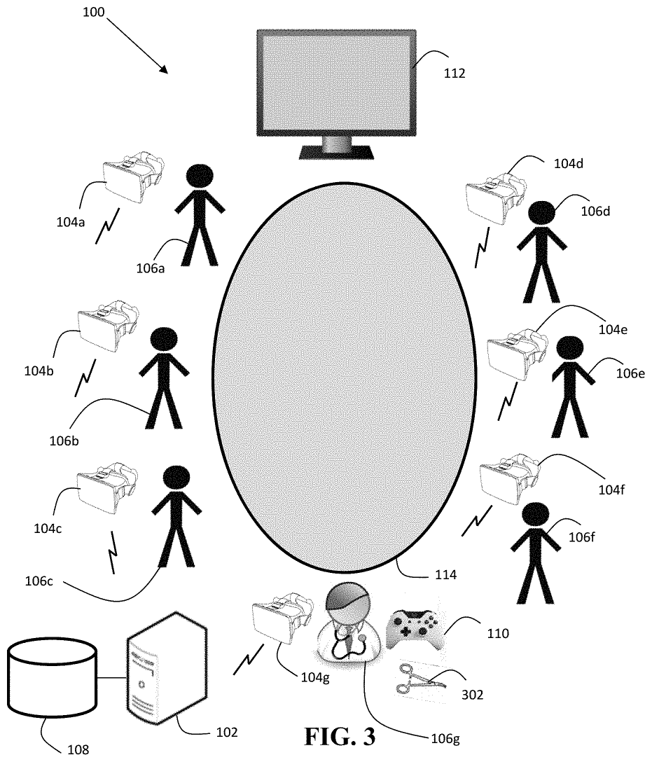

In one example, the VR stadium server 102 may present within the VR stadium 114 a virtual computer (not shown) to which the instructor 106g may navigate to, and browse, a virtual library (not shown) that might be provided by a database or other computer system, locally or remotely. The library may include various types of stored content such as prebuilt SNAP cases that can be retrieved from the VR stadium database 108 for training purposes. For example, an instructor 106g may navigate to the virtual computer, open a virtual library, and select a particular SNAP case for viewing and discussing with other users 106. The instructor 116g may make notes within the SNAP case, or edit the SNAP case as needed, in preparation for particular teaching session, for example. FIG. 2 illustrates an example virtual SNAP computer 200 being used to load an example SNAP case for display on a virtual display 202 within the virtual stadium 114.

In one example, training sessions may be recorded by the VR stadium server 102 and stored in the VR stadium database 108 for later retrieval. For example, an instructor 106g may wish to review the same SNAP case with two separate groups of users 106 at different times and even at different locations and may wish to reuse the same notes, markups, audio recordings, and so on during the second presentation as was created while presenting the first time, while potentially developing additional notes and/or audio recordings in the additional presentation which may also be recorded, if desired. Such presentations may be repeated any number of times, as desired. Thus, the instructor 106g may navigate to the virtual computer 200 and retrieve a recorded session and then begin to train a second, third, or other group using the same session.

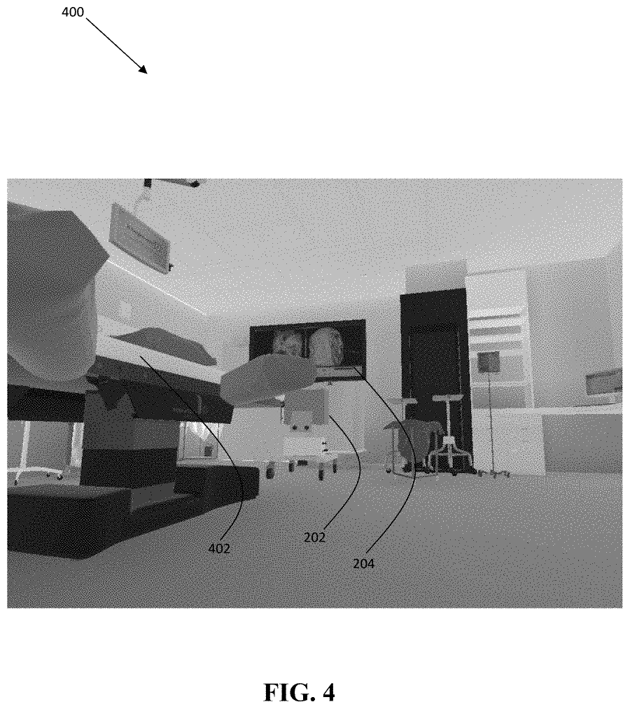

In one example, as illustrated in FIG. 3, the VR stadium system 100 includes one or more tools 302 that can be used by the instructor 106g to simulate procedures. The tools 302 communicate with the VR stadium server 102 in order to translate movements or actions performed by the instructor 106g using the tool 302 in the physical world to the same or similar movements or actions in the VR stadium 114. Such tools 302 may be real medical tools, such as surgical tools, for example, which may be modified to communicate with the system 100. In one example, all of the users 106 may be given the same tool, or the users 106 may take turns using the same tool 106, in order to learn and practice performing the same movement or action. In one example, the VR stadium server 102 translates the movements and actions of the tools 302 into virtual movements and actions within an MD6DM model generated by the VR stadium server 102 based on a SNAP case. In another example, the VR stadium server 102 generates a virtual operating room 400, as illustrated in FIG. 4, which can be navigated to within the VR stadium 114 and interacted with for training and collaboration. For example, once inside the virtual stadium 114, an instructor 106g may direct the users 106 to a virtual patient bed 402 in the virtual operating room 400 where the instructor 106g may demonstrate medical procedures on a virtual patient (not shown) in real time. More particularly, the instructor may make certain movements or actions with the tool 302, which the VR stadium server 102 may translate into corresponding virtual movements or actions within the virtual operating room 400. Users 106 may then observe, via the HDM 104, and learn from the instructor 106g, and in some cases participate themselves in the virtual medical procedure. Virtual representations of the tools 106 may be provided on the displays of the system 100.

In one example, users 106 may be restricted to certain views and perspectives of the virtual operating room and only follow along the same perspective as viewed by the instructor 106g. In another example, users 106 may be free to change their perspective of view of the virtual operating room and of the virtual patient lying on the virtual patient bed 402. For example, via a controller 110 or via motion sensors, the VR stadium server 102 may detect movement and then translate that movement into corresponding movement within the virtual operating room 400. Thus, while an instructor is performing a virtual medical procedure, a user 106 may walk to an opposite side of the patient and view the procedure being performed from a different angle, if the user 106 believes the view from the other angle to be beneficial and educational, for example.

In one example, users 106 may be represented by avatars within the VR stadium 114 so that the users 106 may visualize movement of other users 106, which may enable further interaction and collaboration.

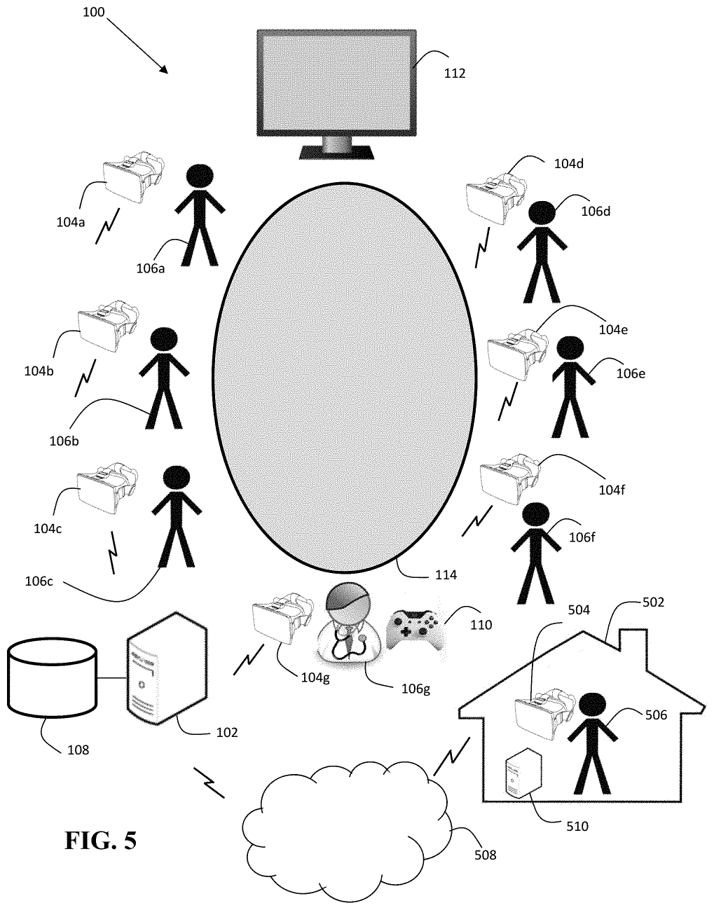

It should be appreciated that the users 106 may or may not all be located in the same room or physical location in order to join a virtual stadium 114. For example, as illustrated in FIG. 5, one or more users 506 may be physically located in a remote location 502 and still participate a training or collaboration session inside the virtual stadium 114, using a remote HDM 504. The remote HDM 504 may communicate with the VR stadium server 102 via the Internet 508, for example, in order to obtain content and to synchronize with the other HDMs 104. Thus, the remote user 506 may see the same content and participate in a training or collaboration session just as the other users 106. In one example, the remote HDM 506 may communicate with a local computing device or server 510, which in turn communicates with the VR stadium server 102. In one example, all of the users may located a physical location different that that of the instructor 106g.

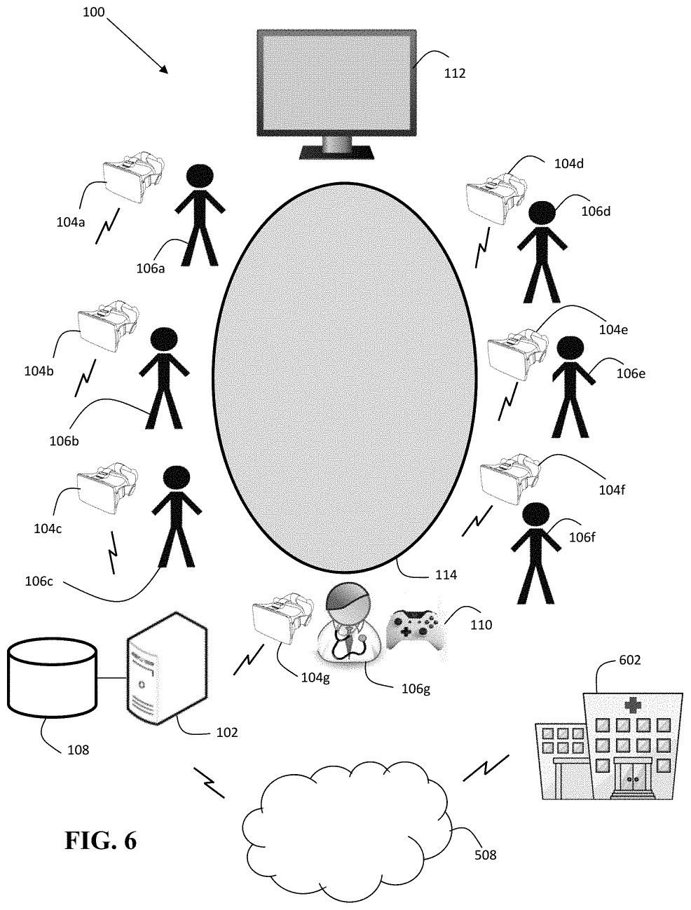

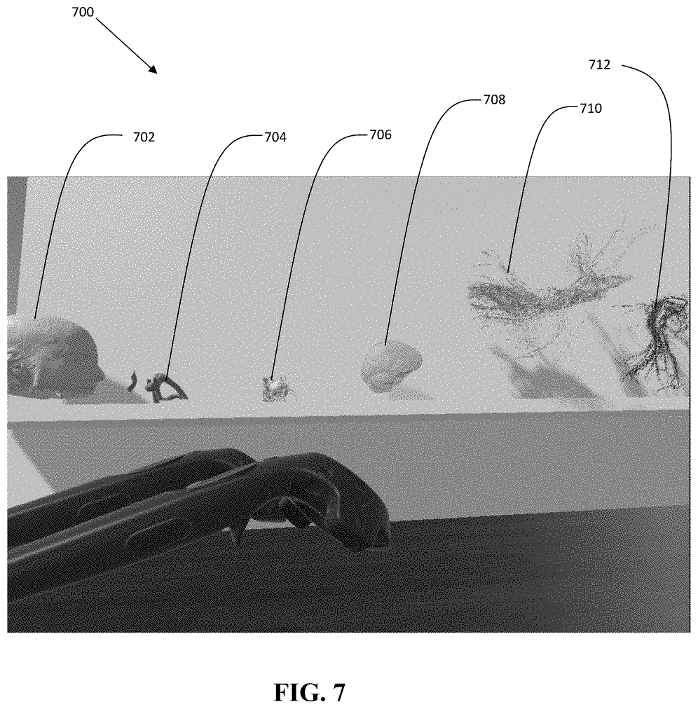

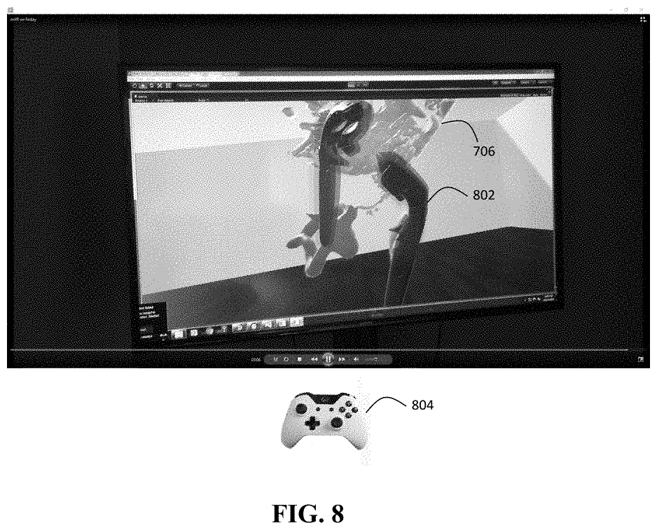

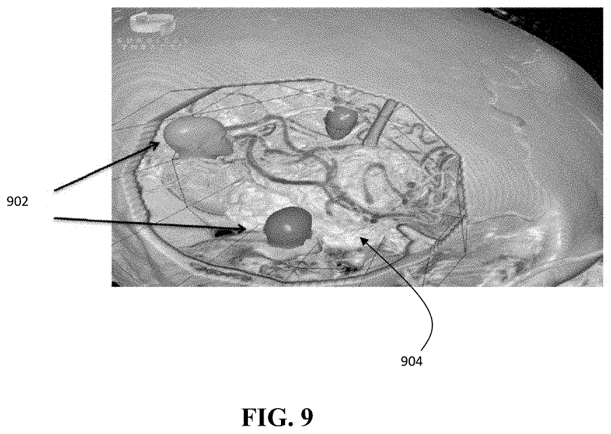

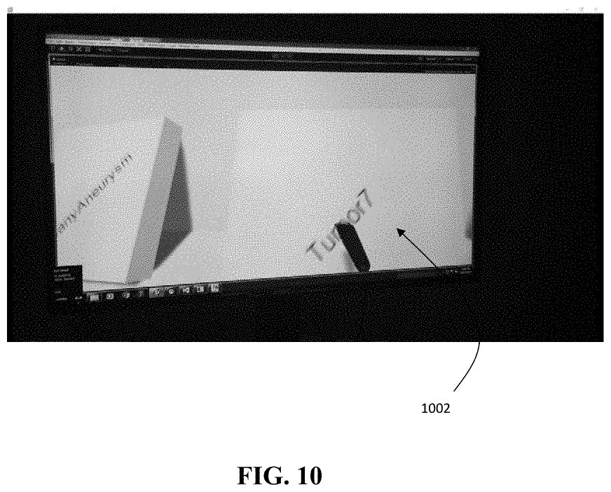

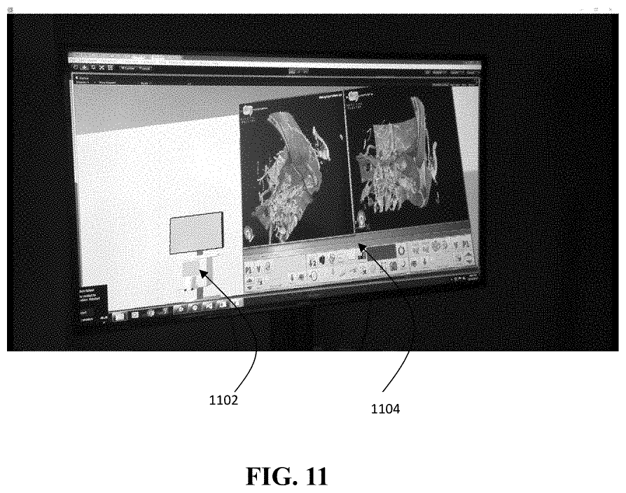

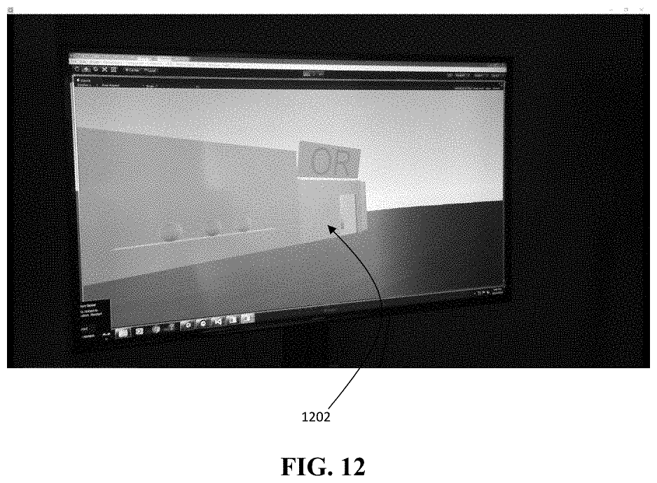

In one example, as illustrated in FIG. 6, the VR stadium system 100 includes a connection to a remote hospital 602 via the internet 508. In particular, the VR stadium server 102 receives a live real-time feed from within a physical operating room inside a remote hospital 602. The live real time feed is then presented by the virtual stadium server 102 to the users 106 via HDMs 104 within the virtual stadium 114. Thus, an instructor 106g may view and discuss with users 106 in real time the details of a procedure being performed for educational purposes, without taking up valuable room inside an operating room. In addition, one or more remote users 506 located in different rooms, different buildings, or even in different geographical locations (such as different states and even different nations) may also utilize the virtual stadium system 100 to provide guidance and support for a physician physically present in the hospital 602 and performing the medical procedure. Thus, real time collaboration is fostered among several medical professionals at various physical locations, enabling collaboration among experts that may be physically located in many different locations.

The live data feed from the hospital 602 may be a real time video feed captured from an endoscope positioned at the patient, for example. The live data feed may also include a VR or AR feed from the perspective of a physician located at the remote hospital 602 wearing an HDM 104 and navigating a virtual MD6DM model via a SNAP computer (not shown) located at the remote hospital 602.

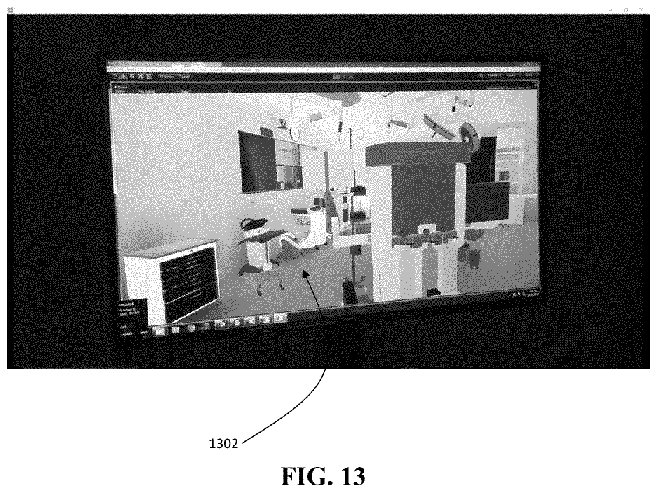

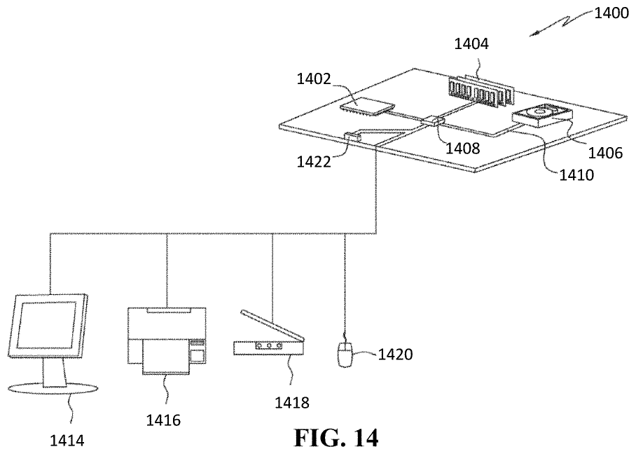

In one example, a user 106 may be able to interact with various 3D models. For example, as illustrated in FIG. 7, the VR stadium 114 may include a virtual 3D model display 700 where a user 106 may navigate to or walk up to and pick up, rotate, examine from different angles, and learn from the various 3D models. In one example, the 3D models may be exported from a SNAP case and be patient specific, which may include images of organs or other tissues obtained using medical imaging procedures that may have occurred earlier in time. In another example, the 3D models may be general models, not applicable to any specific patient. The example 3D model display 700 includes a head 702, an aneurysm 704, a tumor 706, a cortex 708, and DTI tracts 710 and 712. It should be appreciated that, although the example 3D model display 700 illustrated includes a specific set of 3D models, the 3D model display 700 may include any suitable number and types of 3D models which may be based on a particular patient or a generalized model not based on a particular patient.

When pertaining to a particular patient, the 3D models of that patient's organs and tissues are generated from medical images performed on that particular patient, so that the resulting 3D models reflect the actual tissue and organ structures of that particular patient, allowing simulation of medical procedures to be performed as if those procedures were being performed on that particular patient.

The above descriptions may be further appreciated with reference to a specific example scenario in which multiple users log in from a remote location and enter the VR stadium 114 as avatars. Once inside, the users may navigate to the 3D model display 700 and select a model to interact with. The user may also select one or more virtual tools to interact with the model, with such tools possibly being based on real medical tools communicating with the system and displayed as virtual representations of the tools. For example, as illustrated in FIG. 8, a user may select the tumor model 706 to interact with using a virtual tool, 802, or a virtual hand. The virtual hand 802 may be controlled by real world human gestures, for example, using sensors or other similar devices for tracking movement. In another example, the virtual hand 802 may be controlled by a controller 804. It should be appreciated that although the example scenario may be described with reference to specific model, the users may similarly interact with a variety of types of models in preparation for various types of surgical procedures in the VR stadium 114. For example, the VR stadium 114 may be used in connection with preparing for and training to perform surgical procedures in connection with the brain such as an aneurysm or a brain tumor, tumors in other parts of the body, the spinal cord, the heart, and so on.

Once a 3D model is selected, the users may interact with the model by moving it around inside the VR stadium 114, rotating it, and so on. While one of the users (an instructor for example) is interacting with the model, the remaining users may observe the interaction and move around the model. In one example, the remote users may take turns interacting with the model while the remaining users observe the interaction, thus facilitating a virtual collaborative environment. Interacting with the model may include, for example, explaining the model to the other users, asking and answering questions, taking measurements, adding notes to the model, and performing surgical demonstrations, any of which may be recorded for future playback. It should be appreciated that interaction may be facilitated by using other available input tools for converting real world gestures or actions into virtual actions within the VR stadium 114.

In one example, the users may further interact with the selected model by going inside the model 902 with avatars 902 and exploring the inside of the model 902, as illustrated in FIG. 9. Thus, an instructor may be able to zoom in and focus much more closely on very specific internal areas of the model 902 while giving the users an opportunity to navigate around the inside with their respective avatars 902 and observe from their own chosen perspectives.

In another example, users may interact with the selected model by using one or more virtual tools selected from a tool library stored in a database for interacting with the organs or other tissues of the patients. These virtual tools may be representations of real medical tools that communicate with the system, and which the users manipulate in real space to have their virtual representations react similarly in the virtual space. The interaction of the tools with the tissue models is performed in a realistic manner, as described in the '791 patent, such that the a tool model of a user tool (e.g., a surgical tool, probe, implantable medical device, etc.) are shown dynamically interacting with realistic dynamic images of the tissues such that user inputs to input interfaces are used for dynamically manipulating realistic user tool images that are shown dynamically interacting with the realistic images of tissues and organs for realistically simulating a actual medical procedure, such as one on the simulated tissues and organs reflecting the tissues and organs of an actual particular patient, for example. In this way, simulations of medical procedures performed, or to be performed on a particular patient can be simulated for practice, preparation, or educational purposes, for example.

In order to supplement the interactions with the virtual 3D models, the users may also access library resources 1002 for a particular case such as tumor, as illustrated in FIG. 10. The library resources may include videos, books, periodicals, audio recordings, and so on, which may be reviewed and studied virtually among the group of users simultaneous to or in parallel with examining and studying a 3D model. In one example, the users may also retrieve a prebuilt SNAP case from the library and load it on a virtual computer 1102 for display on a virtual display 1104 within the VR stadium 114.

After completing preparation using the 3D models and the library resources, the users may navigate via their respective avatars to a virtual operating room 1202 within the virtual stadium 114 for additional education and perpetration for surgery. In particular, once inside the virtual operating room 1302, a user or group of users may perform a virtual surgical procedure, for which they have been preparing using the 3D models and library resources, on a virtual patient. Additional users may observe the virtual surgical procedure within the virtual operating room 1302. The users have a 360 degree view and access to the virtual patient and can therefore navigate around the patient in order to perform or observe the surgical procedure. The users may speak with one another virtually, via individual microphones, for example, and collaborate inside the virtual operating room 1302 as if the users were all located in the same physical operating room, even though the users may all be dispersed in various remote locations. It should be further appreciated that the virtual operating room 1302 may include various virtual equipment that a user may interact with during the virtual surgical procedure that a user may be accustomed to seeing and using in a physical world operating room, including a SNAP computer and display for displaying a prebuilt SNAP case.

Once preparation for a surgical procedure has completed, remote users may still leverage the virtual stadium 114 in order to be virtually present during the actual surgical procedure, even though the users may be located in various remote locations. The users log into the VR stadium 114 remotely and access, via their respective avatars, a real time 360 degree video and audio feeds streaming from multiple locations inside the physical operating room where the surgical procedure is being performed. Thus, the remote users are able to observe and even collaborate with and assist the surgeons and other medical professional staff present at the physical operating room as if they were themselves physically located in the operating room.

It should be appreciated that all data being communicated within the VR stadium system 100 and with external hospitals 602 can be encrypted and can be made, for example, HIPPA compliant in order to prevent unauthorized access and meet applicable government regulations of respective countries.

FIG. 14 is a schematic diagram of an example computer for implementing the example AVR stadium server 102 of FIGS. 1, 3, 5, and 6. The example computer 1400 is intended to represent various forms of digital computers, including laptops, desktops, handheld computers, tablet computers, smartphones, servers, and other similar types of computing devices. Computer 1400 includes a processor 1402, memory 1404, a storage device 1406, and a communication port 1408, operably connected by an interface 1410 via a bus 1412.

Processor 1402 processes instructions, via memory 1404, for execution within computer 800. In an example embodiment, multiple processors along with multiple memories may be used.

Memory 1404 may be volatile memory or non-volatile memory. Memory 1404 may be a computer-readable medium, such as a magnetic disk or optical disk. Storage device 1406 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 1404 or storage device 1406.

Computer 1400 can be coupled to one or more input and output devices such as a display 1414, a printer 1416, a scanner 1418, and a mouse 1420.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1-18. (canceled)

19. A method for facilitating a collaboration over a medical procedure, said method comprising the steps of:
Providing a medical collaboration system comprising: a computer server comprising one or more processors, one or more computer-readable tangible storage devices, at least one database, and at least one program module stored on at least one of the one or more storage devices for execution by at least one of the one or more processors;
providing a plurality of head mounted displays each including a three dimensional display and at least one input device associated with each one of the head mounted displays;
configuring, by said server executing software instructions in said at least one program module, one of said head mounted displays for being a lead head mounted display;
configuring, by said server executing software instructions in said at least one program module, a plurality of others of said plurality of head mounted displays for being user head mounted displays;
said server executing software instructions in said at least one program module to accept user inputs from a lead user of the input device of said lead head mounted display to configure each one of the user head mounted displays with a limited range of functionality that may or may not be different for some of said user head mounted displays;
said server executing software instructions in said at least one program module to perform a three-dimensional medical procedure process directed by the user of the lead head mounted display for display in each of the plurality of head mounted displays, said simulation process including the steps of:
providing a realistic three dimensional medical tool model of a real medical tool generated from information about physical properties of said real medical tool stored as data in the at least one database, said medical tool models being controlled in said facilitated medical procedure by the lead user using the input device of said lead head mounted head mounted display,
providing realistic three dimensional anatomical models of each one of a plurality of real anatomical objects, said anatomical models being generated from information about physical properties of respective ones of said real anatomical objects stored as data in the at least one database, and
generating, for display on the lead head mounted display and on the plurality of user head mounted displays realistic visual interactions of said medical tool model with said anatomical models during the facilitated medical procedure based on inputs from the lead user using the input device of said lead head mounted display, wherein said realistic visual interactions are indicative of actual interactions of the real medical tool with the real anatomical objects in an actual medical procedure in the real world;
wherein users of said others of the said head mounted display using respective associated input devices are limited in participating in said facilitated medical procedure based on the limited range of functionality of the respective head mounted displays.

20. The method of claim 19, wherein said real medical tool is a surgical tool.

21. The method of claim 19, wherein said real anatomical objects are organs and/or other tissues of a human being.

22. The method of claim 19, wherein said real anatomical objects are organs and/or other tissues of a particular patient based on medical images taken of that particular patient.

23. The method of claim 19, wherein said facilitated medical procedure simulation is a simulation of a surgical procedure on a patient.

24. The method of claim 23, wherein said simulation of the surgical procedure is provided during an actual surgical procedure that is being performed on a patient in real time.

25. The method of claim 24, wherein the lead user of the lead head mounted display is a surgeon performing at least part of the actual surgical procedure on the patient.

26. The method of claim 19, wherein said collaboration system is configured to accept inputs from the lead user of said lead head mounted display for granting specific permissions to interact with the facilitated medical procedure simulation to one or more users of the user head mounted displays.

27. The method of claim 19, wherein said collaboration system is configured to accept inputs from the lead user of said lead head mounted display that cause the one or more users of the user head mounted displays to view the facilitated medical procedure from the perspective of the lead user of said lead head mounted display.

28. The method of claim 19, wherein said input device associated with the head mounted displays includes a controller separate from the head mounted displays.

29. The method of claim 19, wherein said input device associated with the head mounted displays includes an input device integrated in the head mounted displays.

30. The method of claim 19, wherein said input device associated with the head mounted displays includes an input device configured to track the movement of the respective user's hand(s) and/or a tool held by the user.

31. The method of claim 19, wherein the input device of the lead user includes a motion detection input device configured to detect a motion of one or both hands of the lead user and/or a motion of a tool held by the lead user for controlling said interactions.

32. The method of claim 19, wherein the input device of the lead user includes an input device configured to detect a motion of a surgical tool held by the lead user for controlling said interactions.

33. The method of claim 19, wherein said facilitated medical procedure includes providing a capability of the lead user to move around the anatomical models, pick up the anatomical models for closer inspection, move around a room, perform procedures on the anatomical models inside the room, and/or engage with content in a virtual library provided by the server.

34. The method of claim 19, wherein the users of at least some of the user head mounted displays are located a geographic distance remotely from the user of the lead head mounted display.

35. A method for facilitating a collaboration over a medical procedure, said method comprising the steps of:
converting medical images of a particular patient into data representing realistic three-dimensional models of the particular patient's organs and other tissues;
providing a medical collaboration system comprising: a computer server comprising one or more processors, one or more computer-readable tangible storage devices, at least one database, and at least one program module stored on at least one of the one or more storage devices for execution by at least one of the one or more processors;
storing said data representing realistic three-dimensional models of the particular patient's organs and other tissues in the at least one database including physical properties of the organs and other tissues;
storing said data representing realistic three-dimensional models of a real surgical tool including physical properties of the real surgical tool in the at least one database;
providing a plurality of head mounted displays each including a three dimensional display and at least one input device associated with each one of the head mounted displays;
configuring, by said server executing software instructions in said at least one program module, one of said head mounted displays for being a lead head mounted display;
configuring, by said server executing software instructions in said at least one program module, a plurality of others of said plurality of head mounted displays for being user head mounted displays;
said server executing software instructions in said at least one program module to accept user inputs from a lead user of the associated input device of said lead head mounted display to configure each one of the user head mounted displays with a limited range of functionality that can be configured to be different for some of said user head mounted displays but not others of said user head mounted displays;
said server executing software instructions in said at least one program module to perform a three-dimensional surgical simulation process directed by the user of the lead head mounted display for display in each of the plurality of user head mounted displays, said simulation process including the steps of:
providing a realistic three dimensional surgical tool model of the real surgical tool generated from said data representing realistic three-dimensional models of a real surgical tool retrieved from the database, said surgical tool model being controlled in said simulation by the lead user using the associated input device of said lead head mounted head mounted display,
providing realistic three dimensional organ and tissue models of the particular patient's organs and other tissues, said organ and tissue models being generated from data representing the realistic three-dimensional models of the particular patient's organs and other tissues retrieved from the database, and
generating, for display on the lead head mounted display and on the plurality of user head mounted displays realistic visual interactions of said surgical tool model with said organ and tissue models in said simulation based on inputs from the lead user using the input device of said lead head mounted display, wherein said realistic visual interactions are indicative of actual interactions of the real surgical tool with the real organs and tissue of the patient;
wherein users of said others of the said head mounted display using respective associated input devices participate in said surgical simulation to a lesser extent than said lead head mounted display based on their configuration.

36. The method of claim 35, wherein said surgical simulation is used during an actual surgical procedure that is being performed on the particular patient in real time.

37. The method of claim 36, wherein the lead user of the lead head mounted display is a surgeon performing at least part of the actual surgical procedure on the particular patient.

38. The method of claim 35-37, wherein said collaboration system is configured to accept inputs from the lead user of said lead head mounted display for granting specific permissions to interact with the simulation to one or more users of the others of the head mounted displays.

39. The method of claim 35, wherein said collaboration system is configured to accept inputs from the lead user of said lead head mounted display that cause the one or more users of the others of the head mounted displays to view the simulation from the perspective of the user of said lead head mounted display.

40. The method of claim 35, wherein said input device associated with the head mounted displays includes an input device configured to track the movement of the respective user's hand(s) and/or a tool held by the respective user.

41. The method of claim 35, wherein the input device of the lead user includes a motion detection input device configured to detect a motion of one or both hands of the lead user and/or a motion of a tool held by the lead user for controlling said interactions.

42. The method of claim 35, wherein the input device of the lead user includes an input device configured to detect a motion of a surgical tool held by the lead user for controlling said interactions.

43. The method of claim 35, wherein the users of at least some of the others of said head mounted displays are located remotely from the lead user of the lead head mounted display.

44. The method of claim 35, wherein the users of at least some of the others of said head mounted displays participate in the surgical simulation using the associated input devices of the respective head mounted displays.

45. The method of claim 35, wherein said simulation includes providing a capability of the lead user to move around a 3D patient model including the patient organs and other tissues, go inside the patient's 3D body, pick up 3D model organs for closer inspection, move around a virtual operating room, perform a virtual surgical procedure inside the virtual operating room, and/or engage with content in a virtual library provided by the server.

46. A method for facilitating a collaboration over a medical procedure, said method comprising the steps of:
converting medical images of a particular patient into data representing realistic three-dimensional models of the particular patient's organs and tissues;
providing a medical collaboration system comprising: a computer server comprising one or more processors, one or more computer-readable tangible storage devices, at least one database, and at least one program module stored on at least one of the one or more storage devices for execution by at least one of the one or more processors;
storing said data representing realistic three-dimensional models of the particular patient's organs and other tissues in the at least one database including physical properties of the organs and other tissues;
storing said data representing realistic three-dimensional models of a real surgical tool including physical properties of the real surgical tool in the at least one database;
providing a plurality of head mounted displays each including a three dimensional display and at least one input device associated with each one of the head mounted displays;
configuring, by said server executing software instructions in said at least one program module, one of said head mounted displays for being a lead head mounted display;
configuring, by said server executing software instructions in said at least one program module, a plurality of others of said plurality of head mounted displays for being user head mounted displays;
said server executing software instructions in said at least one program module to accept user inputs from a lead user using the real surgical tool that is configured as the associated input device of said lead head mounted display to configure each one of the user head mounted displays with a limited range of functionality that may or may not be different for some of said user head mounted displays;
said server executing software instructions in said at least one program module to display a three-dimensional surgical process directed by the lead user of the lead head mounted display for capturing an actual surgical procedure performed in real time at least partly performed by the lead user for display in each of the plurality of head mounted displays, said process including the steps of:
providing a realistic three dimensional surgical tool model of the real surgical tool generated from said data representing realistic three-dimensional models of a real surgical tool retrieved from the database, said tool model being controlled by the lead user using the real surgical tool in the surgical procedure,
providing realistic three dimensional organ and tissue models of the particular patient's organs and other tissues, said organ and tissue models being generated from data representing the realistic three-dimensional models of the particular patient's organs and other tissues retrieved from the database, and
generating, for display on the lead head mounted display and on the plurality of user head mounted displays, realistic visual interactions of said surgical tool model with said organ and tissue models based on inputs from the lead user using the input device of said lead head mounted display, wherein said realistic visual interactions are indicative of the actual interactions of the real surgical tool with the real organs and tissue of the patient during the surgical procedure;
wherein users of said others of the said head mounted display are able to view said surgical process using the head mounted display.

47. The method of claim 46, wherein the users of at least some of the others of said head mounted displays participate in the actual surgical procedure on the particular patient using the associated input devices of the user's respective head mounted displays.

48. The method of claim 46, wherein users of at least some of the others of said head mounted displays are located remotely from the lead user.

49. The method of claim 46, wherein said simulation includes providing a capability of the lead user to move around a 3D patient model including the patient organs and other tissues, go inside the patient's 3D body, pick up 3D model organs for closer inspection, move around a virtual operating room, perform a virtual surgical procedure inside the virtual operating room, and/or engage with content in a virtual library provided by the server.

50. A method for facilitating collaboration over a medical procedure, comprising the steps of:
providing a collaboration system comprising:
a database for storing content representative of a plurality of virtual three-dimensional anatomical models corresponding to a plurality of patients;
a lead head mounted display;
a plurality of participant head mounted displays;
a computer server subsystem comprising one or more processors, one or more computer-readable tangible storage devices; and
program modules stored on at least one of the one or more storage devices including program instructions for execution by at least one of the one or more processors to perform a medical simulation comprising the steps of:
selecting a corresponding one of the plurality of the virtual three-dimensional anatomical models from the database responsive to a request to initiate a collaboration with respect to one of the plurality of patients,
synchronously delivering content representative of the selected virtual three-dimensional anatomical model to the plurality of head mounted displays,
receiving data from the lead head mounted display representative of an interaction with the selected virtual three-dimensional anatomical model for performing a virtual medical procedure configured for the one of the plurality of patients, and
synchronously delivering updated content to the plurality of participant head mounted displays based on the received interaction.

51. The method of claim 50, wherein the content further comprises at least one of a virtual medical library and a virtual operating room, and wherein the computer server subsystem further receives data representative of an interaction with at least one of the virtual medical library and the virtual operating room.

52. The method of claim 50, wherein the program module includes program instructions for execution by the at least one of the one or more processors to receive the data representative of the interaction from a controller, associated with the lead head mounted display, the interaction being representative of a movement with respect to the virtual three-dimensional anatomical model, thereby delivering updated content to the lead head mounted display based on an updated perspective view of the virtual three-dimensional anatomical model associated with the movement, and the fourth program module is further configured to synchronously deliver the same updated perspective of view of the virtual three-dimensional anatomical model associated with the movement to all of the plurality of participant head mounted displays.

53. The method of claim 50, wherein the program module includes program instructions for execution by the at least one of the one or more processors to receive data from one of a plurality of controllers associated with the plurality of participant head mounted displays, the data representative of movement inside the virtual three-dimensional anatomical model, and the fourth program module is further configured to deliver an updated perspective of view of the inside of the virtual three-dimensional anatomical model to the one of the plurality of participant head mounted displays based on the movement represented by the associated one of the plurality of the controllers.

54. The method of claim 50, wherein the plurality of head mounted displays each comprise a sensor for tracking motion, wherein the program module includes program instructions for execution by the at least one of the one or more processors to receive data from the sensor of one of the plurality of head mounted displays representative of motion of the one of the plurality of head mounted displays, and wherein the program module includes program also includes instructions for execution by the at least one of the one or more processors to deliver an updated perspective of view of the virtual three-dimensional anatomical model to the one of the plurality of head mounted displays based on the movement represented by the associated one of the plurality of head mounted displays.

55. The method of claim 50, wherein the program module includes program instructions for execution by the at least one of the one or more processors to generate a plurality of avatars inside the virtual three-dimensional anatomical model representative of the respective plurality of head mounted displays, and wherein the fourth program module for synchronously delivering updated content is further configured to deliver an updated representation of an avatar representative of the movement of the one of the plurality of head mounted displays.

56. The method of claim 50, wherein the program module includes program instructions for execution by the at least one of the one or more processors to receive at least one of a user-generated note, mark, and drawing, and wherein the fourth program module is further configured to synchronously deliver the at least one of the user-generated note, mark, and drawing to the plurality of the head mounted displays.

57. The method of claim 50, said system further comprising at least on microphone, wherein the program module includes program instructions for execution by the at least one of the one or more processors to receive audio input associated with the content, and wherein the fourth program module is further configured to synchronously deliver the audio input with the content to the plurality of the head mounted displays.

58. The method of claim 50, wherein the program module includes program instructions for execution by the at least one of the one or more processors to record the collaboration and storing the collaboration in the database.

59. The method of claim 50, further comprising a tool configured to perform a physical action, wherein the program module includes program instructions for execution by the at least one of the one or more processors to interpret the physical action performed by the tool and to translate the physical action into a corresponding interaction with the content, and wherein the program module also includes program instructions for execution by the at least one of the one or more processors to synchronously deliver the interaction corresponding to the translated physical action to the plurality of the head mounted displays.
